(19) United States
(12) Patent Application Publication
(10) Pub. No.: US 2022/0347158 A1
(43) Pub. Date: Nov. 3, 2022

(54) Methods of Treating Conditions Related to the S1P1 Receptor

(71) Applicant: Arena Pharmaceuticals, Inc., San Diego, CA (US)

(72) Inventors: Thai Curtis Nguyen-Cleary, Carlsbad, CA (US); Lisette Marie Acevedo, Encinitas, CA (US)

(21) Appl. No.: 17/765,619

(22) PCT Filed: Sep. 30, 2020
(86) PCT No.: PCT/US2020/053642

§ 371 Date: Mar. 31, 2022

Related U.S. Application Data
(60) Provisional application No. 62/909,113, filed on Oct. 1, 2019.

(51) Int. Cl. A61K 31/404

(52) U.S. Cl. CPC A61K 31/404

(57) ABSTRACT

Provided are methods of treatment of Crohn's Disease comprising prescribing and/or administering to an individual in need thereof a standard dose of (R)-2-(7-(4-cyclopentyl-3-(trifluoromethyl)pbenzyloxy)-1,2,3,4-tetrahydrocyclopenta[b]indol-3-yl)acetic acid (Compound 1), or a pharmaceutically acceptable salt, hydrate, or solvate thereof.

Crohn's Disease (CD) is a chronic, relapsing and remitting, immune-mediated inflammatory condition that may affect the entire gastrointestinal tract and is associated with an increased risk for colon cancer. CD is distinguished from ulcerative colitis (UC) in that it presents as a more severe and widespread inflammation of the gastrointestinal tract and is characterized by chronic inflammation, mucosal and sub-mucosal ulceration and fibrosis, whereas with UC, inflammation is predominately restricted to the mucosal and occasionally the submucosal layer of the colon.

Treatment for patients with CD is generally focused on symptomatic care and mucosal healing with overall goals of inducing and sustaining clinical remission, improving quality of life, and preventing more severe disease manifestations and complications that require hospitalization and surgical intervention. Treatment of CD includes several major classes of medications: corticosteroids, immunosuppressants (such as thiopurines [azathioprine and mercaptopurine] and methotrexate), biologics (anti-tumor necrosis factor alpha [TNFα] [infliximab, adalimumab, and certolizumab pegol], interleukin-12 and −23 antagonist [ustekinumab], integrin receptor antagonists [vedolizumab]), and antibiotics. Janus kinase (JAK) inhibitors are being explored for use in CD (tofacitinib and filgotinib). Though used in the treatment of IBD more broadly, the anti-inflammatory drug 5-aminosalicylic acid (5-ASA) demonstrates a low efficacy preoperatively and at prevention of CD recurrence in the postoperative setting.

CD is considered neither medically nor surgically “curable,” with clinical, endoscopic, and surgical recurrence reported in 50%, 80%, and 30% of patients, respectively. The surgical burden in CD remains high; a meta-analysis showed that the risk that CD patients will require surgery within 5 years of diagnosis is 33.3%, and by 10 years is 46.6%. Furthermore, 25% of CD patients will require additional intestinal surgery within 5 years of their first surgery, further increasing the financial burden and negative impact on a patient's quality of life.

There remains a great unmet clinical need for new efficacious and safe treatments for CD, as current therapies often provide only transient or marginal symptomatic relief. The present disclosure satisfies this need and provides related advantages as well.

Citation of any reference throughout this application is not to be construed as an admission that such reference is prior art to the present application.

### SUMMARY

Provided is a method of treating an individual with moderately to severely active Crohn's Disease comprising: administering to the individual in need thereof a pharmaceutical dosage form comprising a therapeutically effective amount of (R)-2-(7-(4-cyclopentyl-3-(trifluoromethyl)benzyloxy) -1,2,3,4-tetrahydrocyclopenta[b]indol-3-yl)acetic acid (Compound 1), or a pharmaceutically acceptable salt thereof.

In some embodiments, a compound that is (R)-2-(7-(4-cyclopentyl-3-(trifluoromethyl)benzyloxy) -1,2,3,4-tetrahydrocyclopenta[b]indol-3-yl)acetic acid (Compound 1), or a pharmaceutically acceptable salt thereof, is for use in a method of treatment of Crohn's Disease in an individual, wherein the method of use includes administering an induction dose of the compound to the individual for an induction phase, wherein the induction phase is at least 14 weeks long, and administering a maintenance dose of the compound to the individual for a maintenance phase. In some embodiments, the induction dose comprises an amount equivalent to 3 mg of Compound 1. In some embodiments, the maintenance dose comprises an amount equivalent to 2 mg of Compound 1. In some embodiments, the maintenance phase is at least 38 weeks. In some embodiments, the compound is administered at a frequency of once a day during both the induction phase and the maintenance phase. In some embodiments, the individual has moderately to severely active Crohn's Disease.

A phase 2 study demonstrated improved efficacy for 2 mg of Compound 1 over placebo in subjects with UC. However, a Phase 2 study with another selective S1P receptor modulator (amiselimod) did not result in improved efficacy over placebo in subjects with CD (D'Haens G et al. Amiselimod, a selective S1P receptor modulator in Crohn's disease patients: A proof-of-concept study. J Crohn Colitis. 2019; 13(Suppl):5055-5056). Further, while UC and CD have overlapping features, there are differences in pathophysiology, disease location, and extent of disease that may dictate different doses for the treatment of UC and CD. Described herein is the elucidation of safe and efficacious dosing of Compound 1 for the treatment of CD.

These and other aspects of the invention disclosed herein will be set forth in greater detail as the patent disclosure proceeds.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” means (R)-2-(7-(4-cyclopentyl-3-(trifluoromethyl)benzyloxy) -1,2,3,4-tetrahydrocyclopenta[b]indol-3-yl)acetic acid including crystalline forms thereof.

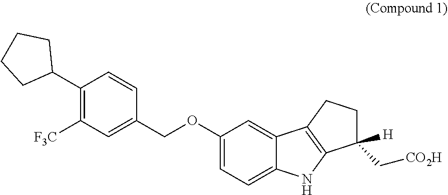 [SMILES: O=C(O)C[C@H]1CCc2c1[nH]c1ccc(OCc3ccc(C4CCCC4)c(C(F)(F)F)c3)cc21]

See PCT patent application, Serial No. PCT/US2009/004265 hereby incorporated by reference in its entirety. As a non-limiting example, Compound 1 may be present as an anhydrous, non-solvated crystalline form as described in WO 2010/011316 (incorporated by reference herein in its entirety). As another non-limiting example, an L-arginine salt of Compound 1 may be present as an anhydrous, non-solvated crystalline form as described in WO 2010/011316 and WO 2011/094008 (each of which is incorporated by reference herein in its entirety). As another non-limiting example, a calcium salt of Compound 1 may be present as a crystalline form as described in WO 2010/011316 (incorporated by reference herein in its entirety).

Compound 1, or a pharmaceutically acceptable salt, solvate, or hydrate thereof, is an orally administered, selective, synthetic sphingosine 1-phosphate (S1P) receptor 1, 4, 5 modulator. To date, Compound 1, or a pharmaceutically acceptable salt, solvate, or hydrate thereof, has been found to be safe and well-tolerated in adult subjects treated at various doses. Its safety and tolerability have been evaluated in Phase 1 studies with healthy adult subjects at single doses up to 5 mg and repeated doses up to 4 mg once daily (“QD” or “qd”). In a Phase 2 dose-ranging study in UC patients, treatment with 2 mg QD for 12 weeks led to clinically meaningful and statistically significant endoscopic and symptomatic improvements versus placebo. Sustained beneficial effects of were observed for up to 46 weeks in the subsequent open-label extension study.

ADMINISTERING: As used herein, “administering” means to provide a compound or other therapy, remedy, or treatment such that an individual internalizes a compound.

CO-ADMINISTER: As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

PRESCRIBING: As used herein, “prescribing” means to order, authorize, or recommend the use of a drug or other therapy, remedy, or treatment. In some embodiments, a health care practitioner can orally advise, recommend, or authorize the use of a compound, dosage regimen or other treatment to an individual. In this case the health care practitioner may or may not provide a prescription for the compound, dosage regimen, or treatment. Further, the health care practitioner may or may not provide the recommended compound or treatment. For example, the health care practitioner can advise the individual where to obtain the compound without providing the compound. In some embodiments, a health care practitioner can provide a prescription for the compound, dosage regimen, or treatment to the individual. For example, a health care practitioner can give a written or oral prescription to an individual. A prescription can be written on paper or on electronic media such as a computer file, for example, on a hand-held computer device. For example, a health care practitioner can transform a piece of paper or electronic media with a prescription for a compound, dosage regimen, or treatment. In addition, a prescription can be called in (oral), faxed in (written), or submitted electronically via the internet to a pharmacy or a dispensary. In some embodiments, a sample of the compound or treatment can be given to the individual. As used herein, giving a sample of a compound constitutes an implicit prescription for the compound. Different health care systems around the world use different methods for prescribing and/or administering compounds or treatments and these methods are encompassed by the disclosure.

A prescription can include, for example, an individual's name and/or identifying information such as date of birth. In addition, for example, a prescription can include: the medication name, medication strength, dose, frequency of administration, route of administration, number or amount to be dispensed, number of refills, physician name, physician signature, and the like. Further, for example, a prescription can include a DEA number and/or state number.

A healthcare practitioner can include, for example, a physician, nurse, nurse practitioner, or other related health care professional who can prescribe or administer compounds (drugs) for the treatment of a condition described herein. In addition, a healthcare practitioner can include anyone who can recommend, prescribe, administer, or prevent an individual from receiving a compound or drug including, for example, an insurance provider.

PREVENT, PREVENTING, OR PREVENTION: As used herein, the term “prevent,” “preventing”, or “prevention,” such as prevention of a particular disorder or the occurrence or onset of one or more symptoms associated with the particular disorder and does not necessarily mean the complete prevention of the disorder. For example, the term “prevent,” “preventing” and “prevention” means the administration of therapy on a prophylactic or preventative basis to an individual who may ultimately manifest at least one symptom of a disease or condition but who has not yet done so. Such individuals can be identified on the basis of risk factors that are known to correlate with the subsequent occurrence of the disease. Alternatively, prevention therapy can be administered without prior identification of a risk factor, as a prophylactic measure. Delaying the onset of at least one symptom can also be considered prevention or prophylaxis.

TREAT, TREATING, OR TREATMENT: As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting, even if the severity of each is less than when experienced alone.

INTOLERANCE: As used herein, “intolerance” means significant toxicities and/or tolerability issues that led to a reduction in dose or discontinuation of the medication. “Intolerance” can be replaced herein with the term “unable to tolerate.”

ADVERSE EVENT: As used herein, an “adverse event” is an untoward medical occurrence that is associated with treatment with Compound 1 or a pharmaceutically acceptable salt, solvate, or hydrate thereof. In one embodiment, an adverse event is selected from: leukopenia, constipation, diarrhea, nausea, abdominal pain, neutropenia, vomiting, back pain, and menstrual disorder. In one embodiment, an adverse event is heart block, for example, a first-degree atrioventricular heart block. In one embodiment, an adverse event is an acute heart rate reduction. In one embodiment, an adverse event is an abnormal pulmonary function test finding, such as an FEV1 below 80%, FVC. In one embodiment, an adverse event is an abnormal liver function test, such as an elevated ALT & AST>2X ULN. In one embodiment, an adverse event is macular edema.

IN NEED OF TREATMENT and IN NEED THEREOF: As used herein, “in need of treatment” and “in need thereof” when referring to treatment are used interchangeably to mean a judgment made by a caregiver (e.g. physician, nurse, nurse practitioner, etc.) that an individual requires or will benefit from treatment. This judgment is made based on a variety of factors that are in the realm of a caregiver's expertise, but that includes the knowledge that the individual is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

INDUCTION DOSE: As used herein, “induction dose” refers to the first dose of Compound 1, or a salt thereof, which is, in some embodiments, larger in comparison to the maintenance dose. The induction dose can be a single dose or, alternatively, a set of doses. In some embodiments, the induction dose is equivalent to the maintenance dose. In some embodiments, the induction dose is smaller than the maintenance dose. In some embodiments, the induction dose is larger than the maintenance dose. The induction dose is often used to bring the drug in the body to a steady state amount, and may be used to achieve maintenance drug levels quickly. In some embodiments, an induction dose is subsequently followed by administration of a smaller dose of Compound 1, i.e., the maintenance dose. The induction dose is administered during the induction phase of therapy. In one embodiment, the induction dose is at least twice the given amount of the maintenance dose. In another embodiment, the induction dose is approximately 1.1 to approximately 1.5 times the given amount of the maintenance dose. In another embodiment, the induction dose is less than the maintenance dose.

MAINTENANCE DOSE: As used herein, “maintenance dose” is the amount of Compound 1, or a salt thereof, taken by a subject to maintain or continue a desired therapeutic effect. A maintenance dose is administered subsequent to the induction dose. A maintenance dose can be a single dose or, alternatively, a set of doses. In some embodiments, maintenance doses are smaller than the induction dose and can be equal to each other when administered in succession In some embodiments, the maintenance dose is equivalent to the induction dose. The maintenance dose is administered during the maintenance phase of therapy. In still another embodiment, the maintenance dose is administered at least two weeks following the induction dose. In still another embodiment, the maintenance dose is administered about 14 weeks following the induction dose. In still another embodiment, the maintenance dose is administered about 20 weeks following the induction dose.

INDIVIDUAL: As used herein, “individual” means any human. In some embodiments, a human individual is referred to a “subject” or “patient.”

ACUTE HEART RATE REDUCTION: As used herein, “acute heart rate reduction” means a heart rate decrease from normal sinus rhythm of, for example, 10 or more beats per minute (bpm), such as less than about 5 bpm, e.g., less than about 4 bpm or less than about 3 bpm or less than 2 bpm, that is maximal within a few hours, for example 1-3 hours, after drug administration, and thereafter the heart rate returns towards the pre-dose value.

NORMAL SINUS RHYTHM: As used herein, “normal sinus rhythm” means the sinus rhythm of the individual when not undergoing treatment. The evaluation of normal sinus rhythm is within the ability of a physician. A normal sinus rhythm will generally give rise to a heart rate in the range from 60-100 bpm.

DOSE: As used herein, “dose” means a quantity of Compound 1, or a pharmaceutically acceptable salt, solvate, or hydrate thereof, given to the individual for treating or preventing the disease or disorder at one specific time.

STANDARD DOSE: As used herein, “standard dose” means the dose of Compound 1, or a pharmaceutically acceptable salt, solvate, or hydrate thereof, that is given to the individual for treating or preventing the disease or disorder. The target dose may vary depending on the nature and severity of the disease to be treated.

THERAPEUTICALLY EFFECTIVE AMOUNT: As used herein, “therapeutically effective amount” of an agent, compound, drug, composition or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician. In some embodiments, the therapeutically effective amount is the standard dose.

MILDLY ACTIVE CROHN′S DISEASE: As used herein, “mildly active Crohn's Disease” means Crohn's Disease characterized by a Crohn's Disease Activity Index (CDAI score) of ≥150 and ≤220. These patients are typically ambulatory and tolerating an oral diet. They have <10 percent weight loss and no symptoms of systemic disease such as fever, tachycardia, abdominal tenderness, and no signs or symptoms of obstruction.

MODERATELY TO SEVERELY ACTIVE CROHN′S DISEASE: As used herein, “moderately to severely active Crohn's Disease” means Crohn's Disease characterized by:
Crohn's Disease Activity Index (CDAI score) of ≥220 and ≤450, and
Unweighted average worst daily abdominal pain (AP) score ≥2 or unweighted average daily loose/watery stool frequency (SF) score ≥4, and
Simple Endoscopic Score in Crohn's disease (SES-CD) of ≥6 or SES-CD ≥4 for subjects with isolated ileal disease.
   This group typically comprises patients who have failed treatment for mild to moderate disease or those patients with prominent symptoms such as fever, weight loss, abdominal pain and tenderness, intermittent nausea or vomiting, or anemia.

SEVERE-FULMINANT CROHN′S DISEASE: As used herein, “severe-fulminant Crohn's Disease” means Crohn's Disease characterized by a Crohn's Disease Activity Index (CDAI score) of ≥450.

CLINICAL REMISSION: As used herein, “clinical remission” with respect to Crohn's Disease means:
Clinical remission APSF (abdominal pain (AP) and loose/watery stool frequency (SF)): Unweighted average worst daily AP score ≤1 (using a 4-point scale; i.e., 0 [none] to 3 [severe]) and unweighted average daily loose/watery (Bristol Stool Form Scale [BSFS] type 6 or 7) SF score ≤3; or
Clinical remission CDAI: CDAI <150; or
Endoscopic remission: SES-CD ≤4 and at least 2-point reduction from baseline with no sub-score >1.

CLINICAL RESPONSE: As used herein, “clinical response” with respect to Crohn's Disease means:
Clinical response APSF: Achieve clinical remission APSF or ≥35% decrease from baseline in unweighted average worst daily AP score and/or ≥60% decrease from baseline in unweighted average daily loose/watery SF score. The unweighted AP and SF scores do not have the CDAI weighting factor applied; or
Clinical response CDAI: Achieve clinical remission CDAI or ≥100-point decrease from baseline in CDAI; or
Clinical response APSF-30: Achieve clinical remission APSF or ≥30% decrease from baseline in unweighted average worst daily AP score and/or ≥30% decrease from baseline in unweighted average daily loose/watery SF score; or
Clinical response CDAI-70: Achieve clinical remission CDAI or ≥70-point decrease from baseline in CDAI; or
Endoscopic response: ≥50% decrease from baseline in SES-CD.

CLINICAL IMPROVEMENT: As used herein, “clinical improvement” with respect to Crohn's Disease means:
Endoscopic improvement: ≥50% decrease in SES-CD.

INADEQUATE RESPONSE (PRIMARY NON-RESPONSE): As used herein, “inadequate response” with respect to Crohn's Disease means signs and symptoms of persistently active disease despite completing an induction regimen at dosage per product label or institutional standard of care.

LOSS OF RESPONSE (SECONDARY NON-RESPONSE): As used herein, “loss of response” with respect to Crohn's Disease means recurrence of signs and symptoms of active disease despite being on a maintenance regimen per institutional standard of care following prior clinical benefit. Discontinuation despite clinical benefit does not qualify as having failed or being intolerant to therapy.

CD-PRO: As used herein, “CD-PRO” is a validated instrument designed to assess the signs, symptoms, and impact of CD through 6 modules: Module 1 (Bowel Signs and Symptoms), Module 2 (Abdominal Symptoms), Module 3 (Systemic Symptoms), Module 4 (Coping Strategies), Module 5 (Daily Life Impact), and Module 6 (Emotional Impact). See Higgins (2018) J. Patient Rep Outcomes 2(1):24.

PRO2: As used herein, “PRO2” means a patient-reported outcome based on the SF and AP components of the CDAI. See Khanna (2015) Aliment Pharmacol Ther. 41(1):77-86.

THE INFLAMMATORY BOWEL DISEASE QUESTIONNAIRE: As used herein, the “Inflammatory Bowel Disease Questionnaire” (IBDQ) is a validated, 32-item questionnaire used to assess health-related quality of life in subjects with IBD (UC and CD). Response to each of the questions is graded from 1 to 7 with overall score ranging from 32 (very poor health-related quality of life) to 224 (perfect health-related quality of life).

ABDOMINAL PAIN NUMERICAL RATING SCALE: As used herein, the “abdominal pain numerical rating scale” (NRS) is a single item that measures the “worst abdominal pain in the past 24 hours” using an 11-point NRS ranging from zero (no pain) to 10 (pain as bad as can imagine).

MEDICAL OUTCOMES STUDY 36 ITEM SHORT FORM HEALTH SURVEY (SF-36): As used herein, the “SF-36” is a 36-item, subject-reported survey of subject health. The SF-36 consists of 36 questions measuring 8 health domains: physical functioning, bodily pain, role limitations due to physical problems, role limitations due to emotional problems, general health perceptions, mental health, social function, and vitality. The subject's responses are solicited using Likert scales that vary in length, with 3 to 6 response options per item. The SF-36 will be scored using 2 overall summary scores: physical component summary and mental component summary scores.

EUROQOL-5 DIMENSIONS (EQ-5D): As used herein, the “EuroQoL-5 Dimensions (EQ-5D) 5-level version” is a widely used quality of life instrument developed in Europe. The EQ-5D includes one question for each of the five quality of life dimensions: mobility, self-care, usual activities, pain/discomfort, and anxiety/depression. The EQ-5D questionnaire also includes a visual analog scale, by which respondents can report their perceived health status with a grade ranging from 0 (the worst possible health status) to 100 (the best possible health status).

WORK PRODUCTIVITY AND ACTIVITY IMPAIRMENT QUESTIONNAIRE: As used herein, the “Work Productivity and Activity Impairment Questionnaire-Crohn's Disease” (WPAI-CD) consists of 6 questions asking about the effect of CD on the subject's ability to work and perform regular activities.

THE PATIENT GLOBAL IMPRESSION OF CHANGE: As used herein, the “Patient Global Impression of Change” (PGIC) is a two-item scale designed to assess a patient impression of the overall change in CD symptoms and whether the change in CD symptoms is meaningful. This questionnaire includes a 7-point Likert scale and is based on a patient's current CD symptoms.

5-AMINOSALICYLATES: As used herein, “5-aminosalicylates” means a class of drugs that include, for example, CANASA® (mesalamine), COLAZAL® (balsalazide disodium), ASACOL® (mesalamine), DELZICOL® (mesalamine), and DIPENTUM® (olsalazine).

IMMUNOSUPPRESSIVES or IMMUNOSUPPRESSIVE AGENTS or IMMUNOSUPPRESANTS: As used herein, “immunosuppressives” or “immunosuppressive agents” or “immunosuppressants” means a class of drugs that include, for example, AZASAN® (azathioprine), IMURAN® (azathioprine), GENGRAF® (cyclosporine), NEORAL® (cyclosporine), and SANDIMMUNE® (cyclosporine).

GLUCOCORTICOSTEROIDS: As used herein, “glucocorticosteroids” means a class of drugs that include, for example, UCERIS® (budesonide); DELTASONE® (prednisone),

MEDROL® (methylprednisolone), and hydrocortisone. Glucocorticosteroids also may be referred to as glucocorticoid or corticosteroids.

TNFα ANTAGONISTS or TNFa INHIBITORS: As used herein, “TNFa antagonists” or “tumor necrosis factor-a antagonists” or “TNFα inhibitors” means a class of drugs that include, for example, SIMPONI® (golimumab), REMICADE® (infliximab), HUMIRA® (adalimumab), and CIMZIA® (certolizumab pegol).

INTEGRIN RECEPTOR ANTAGONISTS: As used herein, “integrin receptor antagonists” means a class of drugs that include, for example, ENTYVIO® (vedolizumab).

PHARMACEUTICAL COMPOSITION: As used here, “pharmaceutical composition” means a composition comprising at least one active ingredient, such as Compound 1; including but not limited to, salts, solvates, and hydrates of Compound 1, whereby the composition is amenable to investigation for a specified, efficacious outcome. Those of ordinary skill in the art will understand and appreciate the techniques appropriate for determining whether an active ingredient has a desired efficacious outcome based upon the needs of the artisan.

AGONIST: As used herein, “agonist” means a moiety that interacts with and activates a G-protein-coupled receptor, such as the S1P₁ receptor, such as can thereby initiate a physiological or pharmacological response characteristic of that receptor. For example, an agonist activates an intracellular response upon binding to the receptor or enhances GTP binding to a membrane. In certain embodiments, an agonist of the invention is an S 1P₁ receptor agonist that is capable of facilitating sustained S1P₁ receptor internalization (see e.g., Matloubian et al., Nature, 427, 355, 2004).

ANTAGONIST: As used herein, “antagonist” means a moiety that competitively binds to the receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist.

HYDRATE: As used herein, “hydrate” means a compound of the invention or a salt thereof, that further includes a stoichiometric or non-stoichiometric amount of water bound by non-covalent intermolecular forces.

SAFETY POPULATION: As used herein, “safety population” means all randomized subjects who received study medication.

SOLVATE: As used herein, “solvate” means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

It is understood that when the phrase “pharmaceutically acceptable salts, solvates and hydrates” or the phrase “pharmaceutically acceptable salt, solvate, or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound lthat are salts, it embraces pharmaceutically acceptable solvates and/or hydrates of such salts.

It will be apparent to those skilled in the art that the dosage forms described herein may comprise, as the active component, either Compound 1 or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

When an integer is used in a method disclosed herein, the term “about” can be inserted before the integer.

Throughout this specification, unless the context requires otherwise, the word “comprise”, or variations such as “comprises” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps, or group of compositions of matter shall be taken to encompass one and a plurality (i.e. one or more) of those steps, compositions of matter, groups of steps, or groups of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination. For example, a method that recites prescribing and/or administering Compound 1 or a pharmaceutically acceptable salt, solvate, or hydrate thereof can be separated into two methods; one method reciting prescribing Compound 1 or a pharmaceutically acceptable salt, solvate, or hydrate thereof and the other method reciting administering Compound 1 or a pharmaceutically acceptable salt, solvate, or hydrate thereof. In addition, for example, a method that recites prescribing Compound 1 or a pharmaceutically acceptable salt, solvate, or hydrate thereof and a separate method of the invention reciting administering Compound 1 or a pharmaceutically acceptable salt, solvate, or hydrate thereof can be combined into a single method reciting prescribing and/or administering Compound 1 or a pharmaceutically acceptable salt, solvate, or hydrate thereof.

Provided is a method of treating an individual with moderately to severely active Crohn's Disease comprising: administering to the individual in need thereof a pharmaceutical dosage form comprising a therapeutically effective amount of (R)-2-(7-(4-cyclopentyl-3-(trifluoromethyl)benzyloxy) -1,2,3,4-tetrahydrocyclopenta[b]indol-3-yl)acetic acid (Compound 1), or a pharmaceutically acceptable salt, hydrate, or solvate thereof.

In some embodiments, the pharmaceutical dosage form is administered once daily to the individual.

In some embodiments, the individual is administered an amount equivalent to about 0.5 to about 5.0 mg of Compound 1. In some embodiments, the individual is administered an amount equivalent to 2 mg of Compound 1. In some embodiments, the individual is administered an amount equivalent to 2.25 mg of Compound 1. In some embodiments, the individual is administered an amount equivalent to 2.5 mg of Compound 1. In some embodiments, the individual is administered an amount equivalent to 2.75 mg of Compound 1. In some embodiments, the individual is administered an amount equivalent to 3 mg of Compound 1.

In some embodiments, the individual is administered an amount equivalent to 2 mg of Compound 1 for a first time period and subsequently an amount equivalent to 3 mg of Compound 1 for a second time period. In some embodiments, the first time period is at least one month, such as one month, two months, three months, four months, etc. In some embodiments, the first time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, etc. In some embodiments, the second time period is at least one month, such as one month, two months, three months, four months, etc. In some embodiments, the second time period is at least one week, such as one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, etc. In some embodiments, the second time period is indefinite, e.g., chronic administration.

In some embodiments, the standard dose is administered without titration. In some embodiments, the standard dose is administered without titration; and the individual does not experience a severe related adverse event. In some embodiments, the standard dose is administered without requiring titration to avoid first-dose effect seen with other S1P receptor modulators.

In some embodiments, if the individual is also being administered glucocorticosteroids, the method further comprises reducing the amount of glucocorticosteroid being administered to the individual. In some embodiments, if the individual is also being administered more than 10 mg/day prednisone or equivalent, the method further comprises reducing the daily dose of prednisone or equivalent by 5 mg/week until receiving 10 mg/day, and then continue reducing at 2.5 mg/week until the daily dose has been reduced to 0 mg/day. In some embodiments, if the individual is also being administered less than or equal to 10 mg/day prednisone or equivalent, the method further comprises reducing the daily dose of prednisone or equivalent by 2.5 mg/week until the daily dose has been reduced to 0 mg/day. In some embodiments, if the individual is also being administered budesonide, the method further comprises reducing the daily dose of budesonide by 3 mg every three weeks until the daily dose has been reduced to 0 mg/day.

In some embodiments, the dosage form is administered under fasted conditions. In some embodiments, the dosage form is administered under fed conditions.

In some embodiments, the method is non-gender specific.

In some embodiments, the individual is also being administered one or more agents independently chosen from:
glucocorticosteroids,
immunosuppressants, such as 6-mercaptopurine, azathioprine, cyclosporine, or methotrexate,
biologics, such as anti-tumor necrosis factor-alpha therapies, e.g., adalimumab, certolizumab, infliximab, or biosimilars thereof; anti-integrin therapies, such as natalizumab or vedolizumab; or anti-interleukin-12 or interleukin-23 therapies, such as ustekinumab, and/or
other medicines for the treatment of Crohn's Disease such as acetaminophen, antibiotics, or loperamide.

In some embodiments, the individual was previously administered at least one agent selected from: a TNFa antagonist, an integrin antagonist, and an immunosuppressive agent. In some embodiments, the individual had an inadequate response with, lost response to, or was intolerant to the at least one agent.

In some embodiments, the individual has demonstrated an inadequate response to, loss of response to, or intolerance of to at least one agent for the treatment of Crohn's Disease chosen from glucocorticosteroids, immunosuppressants, and biologics. In some embodiments, the individual had demonstrated, over the previous 3-month period, an inadequate response to, loss of response to, or intolerance of at least one agent selected from glucocorticosteroids, immunosuppressants, or biologics. In some embodiments, the individual had demonstrated, over the previous 6-month period, an inadequate response to, loss of response to, or intolerance of at least one agent selected from glucocorticosteroids, immunosuppressants, or biologics. In some embodiments, the individual had demonstrated, over the previous 9-month period, an inadequate response to, loss of response to, or intolerance of at least one agent selected from glucocorticosteroids, immunosuppressants, or biologics. In some embodiments, the individual had demonstrated, over the previous 1-year period, an inadequate response to, loss of response to, or intolerance of at least one agent selected from glucocorticosteroids, immunosuppressants, or biologics. In some embodiments, the individual had demonstrated, over the previous 2-year period, an inadequate response to, loss of response to, or intolerance of at least one agent selected from glucocorticosteroids, immunosuppressants, or biologics. In some embodiments, the individual had demonstrated, over the previous 3-year period, an inadequate response to, loss of response to, or intolerance of at least one agent selected from glucocorticosteroids, immunosuppressants, or biologics. In some embodiments, the individual had demonstrated, over the previous 4-year period, an inadequate response to, loss of response to, or intolerance of at least one agent selected from glucocorticosteroids, immunosuppressants, or biologics. In some embodiments, the individual had demonstrated, over the previous 5-year period, an inadequate response to, loss of response to, or intolerance of at least one agent selected from glucocorticosteroids, immunosuppressants, or biologics.

In some embodiments, the individual is also being administered one or more agents independently chosen from 5-aminosalicyclic acid (5-ASA) compounds, low dose oral corticosteroids, and/or anti-diarrheal medications.

In some embodiments, the method further comprises monitoring for adverse events during the administration of Compound 1, or a pharmaceutically acceptable salt, hydrate, or solvate thereof, and optionally, interrupting or terminating the administration of Compound 1, or a pharmaceutically acceptable salt, hydrate, or solvate thereof.

In some embodiments, the treatment further comprises monitoring heart rate during the administration, monitoring pulmonary function during the administration, or monitoring liver function during the administration.

In some embodiments, the treatment further comprises monitoring heart rate during the administration.

In some embodiments, the treatment further comprises monitoring pulmonary function during the administration.

In some embodiments, the treatment further comprises monitoring liver function during the administration.

In some embodiments, the method reduces the incidence and severity of adverse events resulting from the treatment of a condition described herein.

In some embodiments, the adverse event is a serious adverse event.

In some embodiments, the serious adverse event is selected from leukopenia, constipation, diarrhea, nausea, abdominal pain, neutropenia, vomiting, back pain, and menstrual disorder.

In some embodiments, the method results in no serious adverse events.

In some embodiments, the standard dose is administered without substantially inducing an acute heart rate reduction or heart block in the individual.

In some embodiments, the individual has moderately active Crohn's Disease.

In some embodiments, the individual has severely active Crohn's Disease.

In some embodiments, the individual has had inadequate response to conventional therapy.

In some embodiments, the conventional therapy is selected from at least one of corticosteroids, immunosuppressants, and biologics.

In some embodiments, the conventional therapy is selected from at least one of prednisone, budesonide, 6-mercaptopurine, azathioprine, methotrexate, infliximab, adalimumab, or certolizumab pegol.

In some embodiments, the individual is not co-administered a TNFa inhibitor.

In some embodiments, the individual has had CD for ≥3 months.

In some embodiments, the individual has CD involving at least the ileum. In some embodiments, the individual has CD involving at least the colon. In some embodiments, the individual has CD involving at least the ileum and colon. In some embodiments, CD diagnosis has been confirmed by endoscopy. In some embodiments, CD diagnosis has been confirmed by histopathology.

In some embodiments, the individual has a CDAI score ≥200 and ≤450. In some embodiments, the individual has a CDAI score ≥220. In some embodiments, the individual has a CDAI score ≥300. In some embodiments, the individual has a CDAI score ≥200, 210, 220, 225, 230, 240, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490, or 500, or any range of the foregoing. For example, in some embodiments, the CDAI score is ≥220 and ≤300.

In some embodiments, the individual has an unweighted average worst daily AP score ≥2 or unweighted average daily loose/water SF score ≥4.

In some embodiments, the individual has an SES-CD of ≥6. In some embodiments, the individual has an SES-CD ≥4 with isolated ileal disease.

In some embodiments, the individual has evidence of inflammation. In some embodiments, the individual has active Crohn's disease with evidence of inflammation.

In some embodiments, Compound 1 is administered without causing a reduction of more than 6 bpm in heart rate. In some embodiments, Compound 1 is administered without a first-dose effect on heart rate as seen with other S 1P receptor modulators. In some embodiments, Compound 1 is administered without a first-dose effect on AV conduction as seen with other S 1P receptor modulators.

In some embodiments, the method of treatment is for improving endoscopic response. In some embodiments, the method of treatment is for endoscopic improvement, e.g., improving endoscopic appearance of the mucosa.

In some embodiments, the method of treatment is for achieving clinical remission APSF.

In some embodiments, the method of treatment is for decreasing CDAI to <150.

In some embodiments, the method of treatment is for improving clinical response CDAI.

In some embodiments, the method of treatment is for improving clinical response APSF.

In some embodiments, the method of treatment is for improving clinical response CDAI-70.

In some embodiments, the method of treatment is for improving clinical response APSF-30.

In some embodiments, the method of treatment is for decreasing a baseline CDAI score.

In some embodiments, the method of treatment is for decreasing a baseline SES-CD.

In some embodiments, the method of treatment is for increasing a proportion of subjects with clinical response by PRO2.

In some embodiments, the method of treatment is for increasing a proportion of subjects with endoscopic response and clinical remission by PRO2.

In some embodiments, the method of treatment is for decreasing a baseline absolute lymphocyte count.

In some embodiments, the method of treatment is for decreasing a baseline fecal calprotectin (FCP) concentration. In some embodiments, the method of treatment further comprises monitoring the level of fecal calprotectin.

In some embodiments, the method of treatment is for decreasing a baseline in C-reactive protein (CRP) concentration. In some embodiments, the treatment further comprises monitoring the level of C-reactive protein (CRP).

In some embodiments, the method of treatment is for increasing a proportion of subjects who achieve endoscopic remission.

In some embodiments, the method of treatment is for decreasing a baseline in Inflammatory Bowel Disease Questionnaire (IBDQ).

In some embodiments, the method of treatment is for decreasing a baseline in CD-PRO.

In some embodiments, the method of treatment is for decreasing a baseline in SF-36.

In some embodiments, the method of treatment is for decreasing a baseline in EQ-5D.

In some embodiments, the method of treatment is for decreasing a baseline in WPAI-CD.

In some embodiments, the method of treatment is for decreasing a baseline in abdominal pain.

In some embodiments, the method of treatment is for decreasing a baseline in abdominal pain NRS.

In some embodiments, the method of treatment is for improving a baseline in PGIC.

In some embodiments, the method of treatment is for decreasing from baseline the AP or SF subscore of PRO2.

In some embodiments, the method of treatment is for decreasing from baseline the Robarts Histopathology Index Score.

In some embodiments, the method of treatment is for decreasing the time to remission as measured by PRO2 and FCP concentrations.

In some embodiments, the method of treatment is for decreasing the time to response as measured by PRO2 and FCP concentrations.

In some embodiments, the method of treatment is for decreasing the rate of CD-related hospitalizations and surgery.

In some embodiments, the method of treatment is for decreasing the number and/or percentage of draining fistulas.

In some embodiments, the method of treatment is for decreasing the number of draining fistulas.

In some embodiments, the method of treatment is for decreasing the percentage of draining fistulas.

In some embodiments, the method of treatment is for decreasing the number and/or percentage of draining enterocutaneous fistulas.

In some embodiments, the method of treatment is for decreasing the number and/or percentage of draining rectovaginal fistulas.

In some embodiments, the method of treatment is for maintaining fistula closure.

In some embodiments, treating comprises inducing and/or maintaining clinical response; improving endoscopic appearance of the mucosa; and/or inducing and/or maintaining clinical remission.

In some embodiments, treating comprises mucosal healing.

In some embodiments, treating comprises inducing and/or maintaining mucosal healing.

In some embodiments, treating comprises an improvement in the Mucosal Healing Index.

In some embodiments, the treatment is for inducing clinical remission. In some embodiments, the treatment is for maintaining clinical remission. In some embodiments, the treatment is for inducing and maintaining clinical remission.

In some embodiments, the treatment is for inducing clinical response. In some embodiments, the treatment is for maintaining clinical response. In some embodiments, the treatment is for inducing and maintaining clinical response.

In some embodiments, the treatment is for corticosteroid-free remission.

In some embodiments, the treatment is for endoscopic remission.

In some embodiments, treating is reducing a sign and/or symptom of Crohn's disease. In some embodiments, treating is reducing a sign of Crohn's disease. In some embodiments, treating is reducing a symptom of Crohn's disease.

In some embodiments, treating is inducing and/or maintaining clinical remission. In some embodiments, treating is inducing and maintaining clinical remission. In some embodiments, treating is inducing and/or maintaining clinical remission and/or clinical response. In some embodiments, treating is inducing and maintaining clinical remission and clinical response. In some embodiments, treating is inducing clinical remission and/or clinical response. In some embodiments, treating is maintaining clinical remission and/or clinical response. In some embodiments, treating is inducing clinical remission and clinical response. In some embodiments, treating is maintaining clinical remission and clinical response. In some embodiments, treating is reducing signs and/or symptoms of Crohn's Disease. In some embodiments, treating is reducing signs and symptoms of Crohn's Disease. In some embodiments, treating is reducing signs of Crohn's Disease. In some embodiments, treating is reducing symptoms of Crohn's Disease. In some embodiments, treating is reducing signs and symptoms and inducing and maintaining clinical remission of moderately to severely active Crohn's Disease. In some embodiments, treating is reducing symptoms of Crohn's Disease. In some embodiments, treating is reducing signs and symptoms and inducing and maintaining clinical remission of moderately to severely active Crohn's Disease in an individual who has had inadequate response to conventional therapy. In some embodiments, treating is reducing signs and symptoms and inducing and maintaining clinical remission of moderately to severely active Crohn's Disease in an individual who has lost response to or is intolerant to a conventional therapy. In some embodiments, treating is reducing signs and symptoms and inducing and maintaining clinical response in an individual with moderately to severely active Crohn's Disease who has had inadequate response to conventional therapy. In some embodiments, treating is reducing signs and symptoms and inducing and maintaining clinical response in an individual with moderately to severely active Crohn's Disease who has lost response to or is intolerant to a conventional therapy. In some embodiments, treating is inducing and/or maintaining clinical remission and/or mucosal healing. In some embodiments, treating is inducing and maintaining clinical remission and mucosal healing. In some embodiments, treating is inducing and maintaining mucosal healing. In some embodiments, treating is inducing and maintaining clinical remission. In some embodiments, treating is inducing clinical remission. In some embodiments, treating is inducing mucosal healing. In some embodiments, treating is maintaining clinical remission. In some embodiments, treating is maintaining mucosal healing. In some embodiments, treating is achieving and/or sustaining clinical remission in induction responders. In some embodiments, treating is achieving and sustaining clinical remission in induction responders. In some embodiments, treating is achieving clinical remission in induction responders. In some embodiments, treating is sustaining clinical remission in induction responders. In some embodiments, treating is inducing and/or maintaining clinical response. In some embodiments, treating is inducing and maintaining clinical response. In some embodiments, treating is inducing clinical response. In some embodiments, treating is maintaining clinical response. In some embodiments, treating is inducing endoscopic improvement. In some embodiments, treating is maintaining endoscopic improvement. In some embodiments, treating is achieved endoscopic improvement. In some embodiments, treating is improving endoscopic remission. In some embodiments, treating is maintaining endoscopic remission. In some embodiments, treating is inducing histologic healing. In some embodiments, treating is maintaining histologic healing. In some embodiments, treating is improving stool frequency. In some embodiments, treating is maintaining improvement in stool frequency. In some embodiments, treating is improving endoscopic appearance of the mucosa. In some embodiments, treating is maintaining endoscopic improvement of the mucosa. In some embodiments, treating is improving endoscopic appearance of the mucosa during induction. In some embodiments, treating eliminates the need for corticosteroid use. In some embodiments, treating allows for reduced corticosteroid use. In some embodiments, treating allows for the use of a lower dose of a corticosteroid. In some embodiments, treating is achieving corticosteroid-free remission. In some embodiments, treating is sustaining corticosteroid-free remission. In some embodiments, treating comprises corticosteroid taper. In some embodiments, treating comprises weaning off corticosteroids. In some embodiments, treating is improving endoscopic subscore. In some embodiments, treating is maintaining improvement in endoscopic subscore.

In some embodiments, a patient is administered a therapeutically effective amount of Compound 1 or a salt thereof for an induction phase for the treatment of Crohn's Disease. In some embodiments, the induction phase is 14 weeks long. In some embodiments, the induction phase is 20 weeks long. In some embodiments, the induction phase is 8, 9, 10, 11, 12, 13, 15,16, 17, 18, 19, 24, 25, or 30 weeks long. According to some embodiments, during the induction phase the patient receives an induction dose equivalent to 2.0 mg of Compound 1 or a salt thereof.

According to some embodiments, during the induction phase the patient receives an induction dose equivalent to 3.0 mg of Compound 1 or a salt thereof.

During an induction phase, according to some embodiments, the Compound 1 or a salt thereof is administered once a day. During an induction phase, according to some embodiments, the Compound 1 or a salt thereof is administered twice a day. In some embodiments, during an induction phase, the Compound 1 or a salt thereof is administered three or four times a day.

According to some embodiments, during the induction phase, for the first week of the induction phase, the patient receives a first induction dose equivalent to 2.0 mg of Compound 1 or a salt thereof. According to some embodiments, during the induction phase, after the first week of the induction phase (i.e., the second week until the end of the induction phase), the patient receives a second induction dose equivalent to 3.0 mg of Compound 1 or a salt thereof.

In some embodiments, a patient receives a first dose for a first portion of an induction phase that is 14 weeks long, then receives a second dose for a second portion of an induction phase that is 6 weeks long. According to some embodiments, the first dose for the first portion of the induction phase is equivalent to 2.0 mg of Compound 1 or a salt thereof. According to some embodiments, the second dose for the second portion of the induction phase is equivalent to 3.0 mg of Compound 1 or a salt thereof. According to some embodiments, the second dose for the second portion of the induction phase is equivalent to 2.0 mg of Compound 1 or a salt thereof.

In some embodiments, as a result of treatment with Compound 1 or a salt thereof during an induction phase, endoscopic remission or ≥50% decrease from baseline in SES-CD is achieved in a patient in need thereof diagnosed with moderate to severe Crohn's Disease. In some embodiments, as a result of treatment with Compound 1 or a salt thereof after an induction phase, a CDAI of less than 150 is achieved in a patient in need thereof diagnosed with moderate to severe Crohn's Disease. In some embodiments, after treatment during an induction phase as described herein, a patient can achieve a significant change from baseline is SES-CD score. In some embodiments, after treatment during an induction phase as described herein, a patient can achieve a significant change from baseline is CDAI score.

In some embodiments, a patient is administered a therapeutically effective amount of Compound 1 or a salt thereof for a maintenance phase for the treatment of Crohn's Disease. In some embodiments, the maintenance phase is 38 weeks long. In some embodiments, the maintenance phase is 20, 21, 22, 23, 24, 25, 26, 28, 30, 32, 34, 35, weeks long. In some embodiments, the maintenance phase is longer, for example, for 52 weeks or at least 52 weeks, 100 weeks or at least 100 weeks, 208 weeks or at least 208 weeks, or for the life of the patient.

According to some embodiments, during the maintenance phase the patient receives a maintenance dose equivalent to 2.0 mg of Compound 1 or a salt thereof. According to some embodiments, during the maintenance phase, the patient receives a maintenance dose equivalent to 3.0 mg of Compound 1 or a salt thereof.

During a maintenance phase, according to some embodiments, the Compound 1 or a salt thereof is administered once a day. In some embodiments, during maintenance phase, according to some embodiments, the Compound 1 or a salt thereof is administered twice a day. In some embodiments, during a maintenance phase, according to some embodiments, the Compound 1 or a salt thereof is administered three or four times a day.

In some embodiments, as a result of treatment with Compound 1 or a salt thereof after a maintenance phase, clinical remission or a CDAI of less than 150 is achieved in a patient in need thereof diagnosed with moderate to severe Crohn's Disease. In some embodiments, as a result of treatment with Compound 1 or a salt thereof after a maintenance phase, endoscopic remission (SES-CD ≤4 and at least 2-point reduction from baseline with no sub-score >1) or a ≥50% decrease from baseline in SES-CD is achieved in a patient in need thereof diagnosed with moderate to severe Crohn's Disease.

In some embodiments, Compound 1 is not recommended in an individual with active, severe infection. In some embodiments, Compound 1 is not recommended in an individual with an active infection. In some embodiments, Compound 1 is not recommended in an individual with a severe infection. In some embodiments, Compound 1 is not recommended in an individual with an active, severe infection until the infection is controlled. In some embodiments, Compound 1 is not recommended in an individual with an active infection until the infection is controlled. In some embodiments, Compound 1 is not recommended in an individual with a severe infection until the infection is controlled. In some embodiments, administration of Compound 1 is not started during an active infection. In some embodiments, an individual is monitored for infection. In some embodiments, administration of Compound 1 is stopped if an individual develops an infection. In some embodiments, administration of Compound 1 is stopped if infection becomes serious. In some embodiments, administration of Compound 1 is discontinued if an individual develops an infection. In some embodiments, Compound 1 is not administered to an individual with an infection. In some embodiments, Compound 1 is not administered during an active infection. In some embodiments, administration of Compound 1 is not started during active infection; an individual is monitored if an infection develops during administration; and administration is stopped if the infection becomes serious. In some embodiments, an infection is mild. In some embodiments, an infection is moderate. In some embodiments, an infection is severe. In some embodiments, an infection is serious. In some embodiments, an infection is a serious adverse event. In some embodiments, an infection is a respiratory infection.

In some embodiments, Compound 1 is administered without causing a severe adverse event. In some embodiments, Compound 1 is administered without causing a severe adverse event related to heart rate. In some embodiments, Compound 1 is administered without causing a severe adverse event related to heart rate change. In some embodiments, Compound 1 is administered without causing a severe adverse event related to elevated heart rate. In some embodiments, Compound 1 is administered without causing a severe adverse event related to bradycardia. In some embodiments, Compound 1 is administered without causing a severe adverse event related to AV block. In some embodiments, Compound 1 is administered without causing a severe adverse event related to AV conduction. In some embodiments, Compound 1 is administered without causing bradycardia. In some embodiments, Compound 1 is administered without causing AV block. In some embodiments, Compound 1 is administered without causing more than mild decrease in heart rate on first day of treatment (for example, >10 bpm). In some embodiments, Compound 1 is administered without a first-dose effect seen with other S1P receptor modulators. In some embodiments, Compound 1 is administered without a first-dose cardiovascular effect seen with other S1P receptor modulators. In some embodiments, Compound 1 is administered without symptomatic changes in heart rate. In some embodiments, Compound 1 is administered without symptomatic changes in heart rhythm. In some embodiments, Compound 1 is administered without requiring titration to avoid first-dose effect seen with other S1P receptor modulators.

In some embodiments, Compound 1 is administered without increasing a liver function test (LFT). In some embodiments, Compound 1 is administered without causing an elevated LFT. In some embodiments, Compound 1 is administered without increasing ALT. In some embodiments, Compound 1 is administered without increasing AST. In some embodiments, Compound 1 is administered without increasing ALT >3X ULN. In some embodiments, Compound 1 is administered without increasing ALT >2.5X ULN. In some embodiments, Compound 1 is administered without increasing ALT >2X ULN. In some embodiments, Compound 1 is administered without increasing ALT >1.5X ULN. In some embodiments, Compound 1 is administered without increasing AST >3X ULN. In some embodiments, Compound 1 is administered without increasing AST >2.5X ULN. In some embodiments, Compound 1 is administered without increasing AST >2X ULN. In some embodiments, Compound 1 is administered without increasing AST >1.5X ULN. In some embodiments, Compound 1 is administered without increasing bilirubin. In some embodiments, Compound 1 is administered without increasing bilirubin >3X ULN. In some embodiments, Compound 1 is administered without increasing bilirubin >2.5X ULN. In some embodiments, Compound 1 is administered without increasing bilirubin >2X ULN. In some embodiments, Compound 1 is administered without increasing bilirubin >1.5X ULN. In some embodiments, Compound 1 is administered without increasing gamma-glutamyl transferase (GGT). In some embodiments, Compound 1 is administered without increasing GGT >3X ULN. In some embodiments, Compound 1 is administered without increasing GGT >2.5X ULN. In some embodiments, Compound 1 is administered without increasing GGT >2X ULN. In some embodiments, Compound 1 is administered without increasing GGT >1.5X ULN.

In some embodiments, Compound 1 is administered without causing an abnormality in a pulmonary function test. In some embodiments, Compound 1 is administered without causing macular edema.

In some embodiments, the individual has had an inadequate response with, lost response to, been intolerant to, or demonstrated dependence on another agent for the treatment of Crohn's Disease. In some embodiments, the individual has had an inadequate response with the other agent for the treatment of Crohn's Disease. In some embodiments, the individual has lost response to another agent for the treatment of Crohn's Disease. In some embodiments, the individual was intolerant to another agent for the treatment of Crohn's Disease. In some embodiments, the individual requires continuous steroid therapy. In some embodiments, the other agent is at least one agent selected from: a TNFα antagonist, a glucocorticosteroid, an integrin antagonist, and immunosuppressive agent, and an aminosalicylate.

In some embodiments, the individual has had an inadequate response with, lost response to, or been intolerant to a conventional therapy. In some embodiments, the individual has had an inadequate response to conventional therapy. In some embodiments, the individual has lost response to conventional therapy. In some embodiments, the individual has been intolerant to conventional therapy. In some embodiments, the conventional therapy is selected from: at least one agent selected from: a TNFα antagonist, a glucocorticosteroid, an integrin antagonist, and immunosuppressive agent, and an aminosalicylate. In some embodiments, the conventional therapy is selected from at least one of 6-mercaptopurine, azathioprine, cyclosporine, and methotrexate.

In some embodiments, the individual was previously administered a glucocorticosteroid and/or an aminosalicylate. In some embodiments, the individual was previously administered a TNFa antagonist, an integrin antagonist, and/or an immunosuppressive agent.

In some embodiments, the glucocorticosteroid is an oral glucocorticosteroid. In some embodiments, the aminosalicylate is a 5-aminosalicylate. In some embodiments, the integrin antagonist is referred to as an integrin receptor antagonist. In some embodiments, the TNFa antagonist is referred to as a TNFα blocker. In some embodiments, the immunosuppressive agent is referred to as an immunomodulator. In some embodiments, the prior conventional therapy is referred to as prior treatment.

In some embodiments, the Compound 1, or a pharmaceutically acceptable salt, hydrate, or solvate thereof, is administered orally.

In some embodiments, the Compound 1, or a pharmaceutically acceptable salt, hydrate, or solvate thereof, is formulated as a capsule or tablet suitable for oral administration.

In some embodiments, the Compound 1, or a pharmaceutically acceptable salt, hydrate, or solvate thereof, is selected from: Compound 1; a calcium salt of Compound 1; and an L-arginine salt of Compound 1. In some embodiments, the Compound 1, or a pharmaceutically acceptable salt, hydrate, or solvate thereof, is an L-arginine salt of Compound 1. In some embodiments, the Compound 1, or a pharmaceutically acceptable salt, hydrate, or solvate thereof, is an anhydrous, non-solvated crystalline form of an L-arginine salt of Compound 1. In some embodiments, the Compound 1, or a pharmaceutically acceptable salt, hydrate, or solvate thereof, is an anhydrous, non-solvated crystalline form of Compound 1.

In some embodiments, the individual also is administered a therapeutic dose of an oral 5-ASA compound. In some embodiments, the individual also is administered a stable dose of an oral 5-ASA compound.

In some embodiments, the individual also is administered a therapeutic dose of an oral glucocorticosteroid therapy. In some embodiments, the individual also is administered a stable dose of an oral glucocorticosteroid therapy. In some embodiments, the glucocorticosteroid is prednisone, e.g., prednisone at a dose ≤10 mg/day or ≤20 mg/day, or an equivalent steroid. In some embodiments, the glucocorticosteroid is budesonide, e.g., at a dose ≤9 mg/day, or an equivalent steroid.

In some embodiments, the individual also is administered a therapeutic dose of an immunosuppressive agent. In some embodiments, the individual also is administered a therapeutic dose of a thiopurine. In some embodiments, the individual also is administered a therapeutic dose of azathioprine. In some embodiments, the individual also is administered a therapeutic dose of 6-mercaptopurine.

In some embodiments, the individual also is administered a therapeutic dose of thioguanine (also referred to as tioguanine or 6-thioguanine).

In some embodiments, the individual also is administered a therapeutic dose of a probiotic. In some embodiments, the individual also is administered a therapeutic dose of Culturelle. In some embodiments, the individual also is administered a therapeutic dose of Saccharomyces boulardii.

In some embodiments, the individual also is administered a therapeutic dose of an anti-diarrheal. In some embodiments, the individual also is administered a therapeutic dose of loperamide. In some embodiments, the individual also is administered a therapeutic dose of diphenoxylate with atropine.

Also provided are pharmaceutical compositions comprising a standard dose of Compound 1, or, a pharmaceutically acceptable salt, a hydrate or solvate thereof and, optionally, one or more pharmaceutically acceptable carriers. Also provided are pharmaceutical compositions comprising Compound 1, or, a pharmaceutically acceptable salt, a hydrate or solvate thereof, optionally, one or more pharmaceutically acceptable carriers. The carrier(s) must be “acceptable” in the sense of being compatible with the other ingredients of the formulation and not overly deleterious to the recipient thereof.

In some embodiments, Compound 1, or, a pharmaceutically acceptable salt, a hydrate or solvate thereof, is administered as a raw or pure chemical, for example as a powder in capsule formulation.

In some embodiments, Compound 1, or, a pharmaceutically acceptable salt, a hydrate or solvate thereof, is formulated as a pharmaceutical composition further comprising one or more pharmaceutically acceptable carriers.

Pharmaceutical compositions may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. The compounds described herein can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

For oral administration, the pharmaceutical composition may be in the form of, for example, a tablet or capsule. The pharmaceutical composition is preferably made in the form of a dosage unit containing a particular amount of the active ingredient. Examples of such dosage units are capsules, tablets, powders, granules or suspensions, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or encapsulating materials.

In powders, the carrier is a finely divided solid which is in a mixture with the finely divided active component.

In tablets, the active component is mixed with the carrier having the necessary binding capacity in suitable proportions and compacted to the desired shape and size.

The powders and tablets may contain varying percentage amounts of the active compound. A representative amount in a powder or tablet may be from 0.5 to about 90 percent of the active compound. However, an artisan would know when amounts outside of this range are necessary.

Suitable carriers for powders and tablets include magnesium carbonate, magnesium stearate, talc, sugar, lactose, pectin, dextrin, starch, gelatin, tragacanth, methylcellulose, sodium carboxymethyl cellulose, a low melting wax, cocoa butter, and the like. The term “preparation” includes the formulation of the active compound with encapsulating material as carrier providing a capsule in which the active component, with or without carriers, is surrounded by a carrier, which is thus in association with it Similarly, cachets and lozenges are included. Tablets, powders, capsules, pills, cachets, and lozenges can be used as solid forms suitable for oral administration.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets or capsules. Also, the unit dosage form can be a capsule or tablet itself, or it can be the appropriate number of any of these in packaged form.

Further embodiments include the embodiments disclosed in the following Examples, which is not to be construed as limiting in any way.

### EXAMPLES

### Example 1

In a Phase 1, single ascending dose clinical trial in healthy subjects, Compound 1 demonstrated only modest declines in peripheral blood lymphocytes at doses up to 1 mg. In contrast, the 3 mg and 5 mg doses induced a clear dose-responsive decline in the absolute number of peripheral blood lymphocytes. In a Phase 1, multiple ascending dose clinical trial in healthy subjects, up to 21 days of dosing with 2 mg or 3 mg of Compound 1 was safe and well-tolerated and both doses reduced peripheral lymphocyte counts. In another Phase 1 study, 30 adult subjects received 2 mg of Compound 1 qd (Days 1 to 7), then 3 mg of Compound 1 qd (Days 8 to 12), and finally 4 mg of Compound 1 qd (Days 13 to 14). There were no notable safety findings, and subjects tolerated the doses of 2, 3, and 4 mg well. After treatment with Compound 1, lymphocyte reductions of 46% and 65% for Day 8 and Day 15, respectively, were observed.

Together, these Phase 1 studies demonstrated that Compound 1 doses up to 4 mg were well-tolerated with no significant safety findings, and that treatment with 2 mg and 3 mg of Compound 1 resulted in significant reductions in peripheral lymphocyte counts.

### Example 2

Formulations composed of immediate-release, hard gelatin capsules containing an L-arginine salt of Compound 1 were prepared as shown in Table 1.

TABLE 1

| | Formulation | | | | |
|---|---|---|---|---|---|
| | 0.1 mg | 0.35 mg | 0.5 mg | 1 mg | 2 mg |
| L-arginine salt of | 0.14 | 0.48 | 0.69 | 1.38 | 2.76 |
| Compound 1 (mg/capsule) | | | | | |
| Empty capsule weight (mg)* | 38.0 | 61.0 | 61.0 | 61.0 | 61.0 |
| Total capsule target | 38.14 | 61.48 | 61.69 | 62.38 | 63.76 |
| weight (mg)** | | | | | |

*Approximate weight. Based on capsule specification
**Theoretical total weight calculated by combining fill and empty capsule weights together

### Example 3

Formulations composed of immediate-release tablets containing an L-arginine salt of Compound 1 were prepared as shown in Table 2.

TABLE 2

| Tablet Strength | 0.5 mg | 1 mg | 2 mg | 3 mg |
|---|---|---|---|---|
| L-Arg Salt of Compound 1 | 0.69 | 1.381 | 2.762 | 4.143 |
| Mannitol Pearlitol® 100SD | 54.81 | 54.119 | 52.738 | 51.357 |
| Microcrystalline cellulose- | 40 | 40 | 40 | 40 |
| Sodium Starch Glycolate- | 4 | 4 | 4 | 4 |
| Magnesium Stearate | 0.5 | 0.5 | 1 | 0.5 |
| Opadry® II Blue | 4 | 4 | 4 | 4 |
| Total tablet target weight | 104 | 104 | 104 | 104 |

### Example 4

A Phase 2, multicenter, randomized, double-blind, placebo-controlled, parallel-group study will evaluate the efficacy, safety, and tolerability of two doses of Compound 1 versus placebo in subjects with moderately to severely active CD. The study will consist of a screening period to determine subject eligibility, a double-blind induction treatment period (Induction Period), a subsequent extension period (Extension Period), and a Follow-up Period.

### Induction Period

Eligible subjects will be randomized in a double-blinded fashion (1:1:1 ratio) to receive 3 mg of Compound 1, 2 mg of Compound 1, or matching placebo.

### Extension Period

All subjects who complete the Induction Period can enter the Extension Period. Subjects will be assigned to receive 2 mg or 3 mg of Compound 1 in the Extension Period according to their Induction Period treatment and clinical response at the end of the Induction Period.

### Inclusion Criteria

Men or women 18 to 80 years of age
Ability to provide written informed consent or assent and to be compliant with the schedule of protocol assessments
Diagnosed with CD ≥3 months
Have moderately to severely active CD at screening
Demonstrated inadequate response, loss of response to, or intolerance to ≥1 of the following therapies for the treatment of CD:
   a. Oral corticosteroids (e.g., prednisone or its equivalent, budesonide)
   b. Immunosuppressants (e.g., azathioprine [AZA], 6 mercaptopurine [6MP], or methotrexate [MTX])
   c. Tumor necrosis factor alpha (TNFα) antagonists (e.g., infliximab, adalimumab, certolizumab pegol, or biosimilars)
   d. Integrin receptor antagonists (e.g., vedolizumab)
   e. Interleukin 12/23 antagonists (e.g., ustekinumab)
Females of childbearing potential must be nonpregnant
Females of childbearing potential and males must use contraception
Interleukin 12/23 antagonist (e.g., ustekinumab)

### Exclusion Criteria

History of inadequate response (i.e., primary non-response) to agents from ≥2 classes of biologics marketed for the treatment of CD (i.e., TNFα antagonists, interleukin 12/23 antagonists, and integrin receptor antagonists)
Have ulcerative colitis, indeterminate colitis, microscopic colitis, ischemic colitis, radiation colitis, diverticular disease associated colitis, toxic megacolon, or active infectious colitis or test positive for Clostridium difficile toxin at screening
Have functional or post-operative short bowel syndrome or any associated complications that may require surgery or interfere with efficacy assessments
Had surgical treatment for intra-abdominal abscesses ≤8 weeks prior to randomization or surgical treatment for perianal abscesses ≤4 weeks prior to randomization
Had intestinal resection ≤24 weeks prior to randomization or other intra-abdominal surgeries ≤12 weeks prior to randomization
Have an ileostomy or a colostomy
Have a serious infection requiring IV antibiotics/medication(s) ≤4 weeks prior to randomization.
Have primary or secondary immunodeficiency syndromes, opportunistic infection, or infection with HIV, HBV, HCV or tuberculosis (active or latent)
Have a clinically relevant cardiovascular condition or receiving treatments that may affect cardiovascular function
Have active retinopathy or macular edema
Have forced expiratory volume at one second or forced vital capacity <70% of predicted values at screening
Lactating female who is breastfeeding
Any acute illnesses or medical conditions including cognitive impairment and alcohol/drug abuse/dependence, or signs/symptoms suspicious for a serious disease that, in the investigator's opinion, could put the subject at increased risk for safety event(s) or interfere with protocol-specified procedures or adherence with study treatment

### Endpoints

The primary endpoint is the proportion of subjects who achieve endoscopic response at Week 14. The secondary endpoints include:
Proportion of subjects who achieve clinical remission APSF at Week 14
Proportion of subjects who achieve CDAI <150 by visit up to Week 14
Proportion of subjects who achieve clinical response CDAI by visit up to Week 14
Proportion of subjects who achieve clinical response APSF by visit up to Week 14
Proportion of subjects who achieve clinical response CDAI-70 by visit up to Week 14
Proportion of subjects who achieve clinical response APSF-30 by visit up to Week 14
Change from baseline in CDAI score by visit up to Week 14
Change from baseline in SES-CD at Week 14
Proportion of subjects with clinical response by PRO2 at Week 14
Proportion of subjects with endoscopic response and clinical remission by PRO2 at Week 14
Change and percentage change from baseline in absolute lymphocyte count by visit up to Week 14
Change and percentage change from baseline in FCP concentration at Weeks 2, 4, 6, 10, and 14
Change and percentage change from baseline in CRP concentration at Weeks 2, 4, 6, 10, and 14
Proportion of subjects who achieve endoscopic remission at Week 14

### Example 5

A seamless Phase ⅔, multicenter, randomized, double-blind study that comprises five substudies will evaluate the efficacy, safety, and tolerability of Compound 1 in subjects with moderately to severely active CD.

Subjects will be refractory or intolerant to at least one current therapy for CD (e.g., corticosteroids, immunosuppressants, or biologics). Subjects who are refractory or intolerant to corticosteroids and/or immunosuppressants may be either previously exposed to or naïve to biologics. Randomized subjects will be permitted to continue stable doses of 5-ASA compounds, low dose oral corticosteroids, and/or anti-diarrheal medications as background therapy for CD; however, corticosteroid tapering may be required in subjects who continue treatment beyond the Induction Period.

The five substudies are as follows:

Substudy 1: A Phase 2, randomized, double-blind substudy to assess the safety, tolerability, and efficacy of oral Compound 1 in subjects with moderate to severe CD that supports the selection of an induction and maintenance dose(s) for Phase 3. The total duration of this substudy is up to 74 weeks, inclusive of the 28-Day Screening Period, 14-week Induction Period, 52-week Extension Period, and the 4-Week Follow-Up Period. Subjects eligible for this substudy will be randomized in a double-blinded fashion (1:1 ratio) to receive 2 mg or 3 mg of Compound 1 during the Induction Period.

All subjects who complete the Induction Period may enter the Extension Period. Subjects will receive 2 mg or 3 mg of Compound 1 in the Extension Period according to their Induction

Period treatment and clinical response as shown in Table 1.

TABLE 1
Substudy 1-Phase 2 Extension Period Treatment Assignment

| Induction | Met Criteria for Clinical | Extension Period |
|---|---|---|
| Period Treatment | Response at Week 14 | Treatment Assignment |
| 2 mg of | Yes | 2 mg of Compound 1 |
| Compound 1 | No | 2 mg or 3 mg of |
| | | Compound 1 (1:1) |
| 3 mg of | Yes | 3 mg of Compound 1 |
| Compound 1 | No | 3 mg of Compound 1 |

Subjects must meet at least one of the following criteria to be considered a responder (“response criteria”):
Clinical response Crohn's Disease Activity Index (CDAI): clinical remission CDAI OR ≥100-point decrease from baseline in CDAI
   Clinical remission CDAI: CDAI <150
Endoscopic response: endoscopic remission OR ≥50% decrease from baseline in Simple Endoscopic Score in Crohn's Disease (SES-CD)
   Endoscopic remission: SES-CD ≤4 and at least 2-point reduction from baseline with no subscore >1
      Substudy 2: A Phase 2b, randomized, double-blind, placebo-controlled, dose-ranging induction substudy to evaluate Compound 1 as induction therapy and select an induction and maintenance dose(s) for continued evaluation in Phase 3. The total duration of this substudy is up to 28 weeks, inclusive of the 28-Day Screening Period, 14-week Induction Period, 6-week Extended Induction (EI) Period, and the 4-Week Follow-Up Period. Eligible subjects will be randomized in a double-blinded fashion (1:1:1 ratio) to receive 3 mg of Compound 1, 2 mg of Compound 1, or matching placebo. To determine if a proportion of subjects receiving 2 mg or 3 mg of Compound 1 may be delayed responders and may benefit from extended induction treatment, all subjects who do not meet response criteria at Week 14 will enter a 6-week EI Period for a total of up to 20 weeks induction treatment. In the EI Period, subjects will receive 2 mg or 3 mg of Compound 1 according to their Induction Period treatment.
      Substudy 3: A Phase 3, randomized, double-blind, placebo-controlled substudy to evaluate Compound 1 as induction therapy. The total duration of this substudy is up to 28 weeks, inclusive of the 28-Day Screening Period, 14-week Induction Period, 6-week EI Period, and the 4-Week Follow-Up Period. Eligible subjects will be randomized in a double-blind fashion (2:1 ratio) to the selected dose of Compound 1(2 mg or 3 mg) or placebo treatment. To determine if a proportion of subjects receiving 2 mg or 3 mg of Compound 1 may be delayed responders and may benefit from extended induction treatment, all subjects who do not meet response criteria at Week 14 will enter a 6-week EI Period for a total of up to 20 weeks induction treatment. In the EI Period, subjects will receive 2 mg or 3 mg of Compound 1 according to their Induction Period treatment.
      Substudy 4: A Phase 3, randomized, double-blind, placebo-controlled substudy to evaluate Compound 1 as maintenance therapy. The overall duration of this substudy is up to 42 weeks, inclusive of the 38-week Treatment Period and the 4-Week Follow-Up Period. Subjects who complete treatment in Substudy 2 or Substudy 3 and who show clinical improvement may be eligible for Substudy 4. Subjects will be randomized (1:1 ratio) in a double-blind fashion to placebo or Compound 1 for up to 38 weeks; subjects randomized to Compound 1 will receive the same Compound 1 dose (2 mg or 3 mg) they received at the last visit of the parent study or substudy.
      Substudy 5: A long-term extension (LTE) substudy for subjects who complete at least 52 weeks of treatment. The overall duration of this study is up to 212 weeks, inclusive of the 208-week Treatment Period and the 4-Week Follow-Up Period. Subjects from Substudy 4 and from Substudy 1 who complete at least 52 and 66 weeks of treatment, respectively, will be eligible to enter this LTE study with a treatment period of up to 208 weeks and a 4-Week Follow-Up Period.

### Inclusion Criteria

1. Subjects 18 to 80 years of age, inclusive, at the time of consent
2. Ability to provide written informed consent and to be compliant with the schedules of protocol assessments
3. Have CD for ≥3 months prior to randomization, involving the ileum and/or colon, at a minimum; diagnosis may be confirmed at any time in the past by endoscopy and/or histopathology. The screening endoscopy and histopathology reports may serve as source documents for subjects who do not have diagnostic endoscopy reports in their medical chart
4. Have moderately to severely active CD at Screening, defined as:
Crohn's Disease Activity Index (CDAI) score ≥220 and ≤450, AND
Unweighted average worst daily abdominal pain (AP) score ≥2 (using a 4-point scale; i.e., 0 [none] to 3 [severe]) OR unweighted average daily loose/watery stool frequency (SF) (Bristol Stool Form Scale [BSFS] type 6 or 7) score ≥4, AND
Simple Endoscopic Score in Crohn's disease (SES-CD) of ≥6 or SES-CD ≥4 for subjects with isolated ileal disease
5. Demonstrated inadequate response, loss of response to, or intolerance to ≥1 of the following therapies for the treatment of CD:
Oral corticosteroids (e.g., prednisone [or its equivalent] or budesonide)
Immunosuppressants (e.g., azathioprine, 6-mercaptopurine, or methotrexate)

### Exclusion Criteria

1. History of inadequate response (i.e., primary non-response) to agents from ≥2 classes of biologics marketed for the treatment of CD (i.e., TNFα antagonists, interleukin-12/−23 antagonist, and integrin receptor antagonist)
2. Have ulcerative colitis, indeterminate colitis, microscopic colitis, ischemic colitis, radiation colitis, diverticular disease-associated colitis, toxic megacolon, or active infectious colitis or test positive for Clostridioides difficile toxin at Screening
3. Have functional or post-operative short-bowel syndrome (i.e., have >3 small bowel resections) or any associated complications that may require surgery or interfere with efficacy assessments
4. Had surgical treatment for intra-abdominal abscesses ≤8 weeks prior to randomization or surgical treatment for perianal abscesses ≤4 weeks prior to randomization

5. Had intestinal resection ≤24 weeks prior to randomization or other intra-abdominal surgeries ≤12 weeks prior to randomization. Subjects who have undergone previous colonic resection or ileocolectomy must have >25 cm of colon remaining

6. Have an ileostomy or a colostomy
7. Have a serious infection requiring intravenous antibiotic(s)/medication(s) ≤4 weeks prior to randomization
8. Have primary or secondary immunodeficiency syndromes, history of organ transplant, history of an opportunistic infection, history of disseminated herpes simplex or herpes zoster, have or test positive for human immunodeficiency virus, hepatitis B virus, or active hepatitis C virus
9. Lactating female who is breastfeeding

### Dosing and Administration

For Substudy 1, Substudy 2, and Substudy 3, in the 3 mg of Compound 1 treatment groups, for Week 1, patients enrolled will take one tablet containing 2 mg of Compound 1 and one matching placebo tablet orally once daily (qd). From Week 2 onwards, patients enrolled will take one tablet containing 2 mg of Compound 1 and one tablet containing 1 mg of Compound 1 orally qd (for a total daily dosage of 3 mg of Compound 1). Subjects who received the 2 mg dose of Compound 1 in a previous treatment period who are then assigned to the 3 mg dose of Compound 1 will receive 3 mg of Compound 1 from the start of treatment. In the 2 mg of Compound 1 treatment groups, patients enrolled will receive either a) one tablet containing 2 mg of Compound 1 and one matching placebo tablet, or b) one tablet containing 2 mg of Compound 1 if the 3 mg arm is discontinued. In the placebo group, Two matching placebo tablets taken orally qd, or one matching placebo tablet taken orally qd (if the 3 mg dose of Compound 1 is discontinued).

For Substudy 4, in the 3 mg of Compound 1 treatment groups, patients enrolled will take one tablet containing 2 mg of Compound 1 and one tablet containing 1 mg of Compound 1 orally qd (for a total daily dosage of 3 mg of Compound 1). In the 2 mg of Compound 1 treatment groups, patients enrolled will receive either a) one tablet containing 2 mg of Compound 1 and one matching placebo tablet, or b) one tablet containing 2 mg of Compound 1 if the 3 mg arm is discontinued. In the placebo group, two matching placebo tablets taken orally qd, or one matching placebo tablet taken orally qd (if the 3 mg dose of Compound 1 is discontinued).

For Substudy 5, in the 3 mg of Compound 1 treatment groups patients enrolled will take one tablet containing 2 mg of Compound 1 and one tablet containing 1 mg of Compound 1 orally qd (for a total daily dosage of 3 mg of Compound 1). In the 2 mg of Compound 1 treatment groups, patients enrolled will receive either a) one tablet containing 2 mg of Compound 1 and one matching placebo tablet, or b) one tablet containing 2 mg of Compound 1 if the 3 mg arm is discontinued.

Primary and Secondary endpoints will vary based on substudy and follow the definitions below:

Endpoint definitions:
Endoscopic response: endoscopic remission or ≥50% decrease from baseline in SES-CD
Endoscopic remission: SES-CD ≤4 and at least 2-point reduction from baseline with no sub-score >1
Clinical response CDAI: Clinical remission CDAI or ≥100-point decrease from baseline in CDAI
Clinical remission CDAI: CDAI <150
Clinical response PRO2: Clinical remission PRO2 or ≥8-point decrease from baseline in PRO2
Clinical response CDAI-70: Clinical remission CDAI or ≥70-point decrease from baseline in CDAI
Clinical remission PRO2: PRO2 <8
Corticosteroid-free remission: CDAI <150 without receiving corticosteroids for ≥4 weeks prior to Week 52 (for subjects receiving corticosteroids at baseline)

Based on above definitions, subjects in Substudy 2 or Substudy 3 who meet response criteria at Week 14 (i.e., Week 14 responders, defined as subjects who have met at least 1 criterion of endoscopic response, endoscopic remission, clinical response CDAI >100 improvement, or clinical remission CDAI <150) will be eligible to enter Substudy 4 upon completion of the Week 14 Visit. Subjects in Substudy 2 or Substudy 3 who do not meet response criteria at Week 14 based on the above definitions will be eligible for a 6-week EI Period in Substudy 2 or Substudy 3.

### Substudy 1

Primary efficacy endpoints:
Change from baseline in SES-CD score at week 14 and 52
Change from baseline in CDAI score at week 14 and 52 Secondary efficacy endpoints:
Proportion of subjects with endoscopic response at week 14 and 52
Proportion of subjects with clinical remission CDAI at week 14 and 52

### Substudy 2

Primary efficacy endpoint:
Proportion of subjects with endoscopic response at Week 14 Secondary efficacy endpoint:
Proportion of subjects with clinical remission CDAI at Week 14

### Substudy 3

Primary efficacy endpoints:
Proportion of subjects with endoscopic response at Week 14
Proportion of subjects with clinical remission CDAI at Week 14 Secondary efficacy endpoints:
Proportion of subjects with clinical remission PRO2 at Week 14
Proportion of subjects with clinical response CDAI at Week 14
Proportion of subjects with endoscopic response and clinical remission CDAI at Week 14
Proportion of subjects with endoscopic remission at Week 14

### Substudy 4

Primary efficacy endpoints:
Proportion of subjects with clinical remission CDAI at Week 52
Proportion of subjects with endoscopic response at Week 52
   Secondary efficacy endpoints:
Proportion of subjects with clinical remission CDAI at Week 52 among subjects in clinical remission CDAI at Substudy 4 baseline (defined as Week 14 or EI-Week 6 Visit)
Proportion of subjects with endoscopic response at Week 52 among subjects in endoscopic response at Substudy 4 baseline
Proportion of subjects with corticosteroid-free clinical remission CDAI at Week 52 among subjects receiving corticosteroids at Substudy 4 baseline
Proportion of subjects with endoscopic remission at Week 52
Proportion of subjects with clinical remission PRO2 at Week 52

### Substudy 5

Secondary efficacy endpoints:
Proportion of subjects with clinical remission CDAI by visit up to the end of treatment
Proportion of subjects with clinical remission PRO2 by visit up to the end of treatment
Safety will be assessed by vital signs, physical exams including ophthalmic exam with optical coherence tomography, adverse events (AEs), laboratory assessments (e.g., hematology, chemistry, coagulation panel, pregnancy test for women of child-bearing potential only), 12-lead electrocardiogram (ECG), continuous Holter monitoring, and pulmonary function tests and diffusing capacity of the lungs for carbon monoxide where locally available. Safety endpoints:
Incidence of treatment-emergent AEs (TEAEs) and serious AEs (SAEs)
Incidence and severity of laboratory abnormalities
Incidence of clinically significant vital sign, ECG, and Holter abnormalities
Change from baseline in laboratory values (hematology, serum chemistry, coagulation, and urinalysis), ECG, and vital signs
Other uses of the disclosed methods will become apparent to those in the art based upon, inter alia, a review of this patent document.

What is claimed is:

1. A method of treating an individual with Crohn's Disease comprising: administering to the individual in need thereof a pharmaceutical dosage form comprising a therapeutically effective amount of (R)-2-(7-(4-cyclopentyl-3-(trifluoromethyl)benzyloxy)-1,2,3,4-tetrahydrocyclopenta [b]indol-3-yl)acetic acid (Compound 1), or a pharmaceutically acceptable salt thereof.

2. The method of claim 1, wherein the individual has demonstrated an inadequate response to, loss of response to, or intolerance of to at least one agent for the treatment of Crohn's Disease chosen from glucocorticosteroids, immunosuppressants, and biologics.

3. The method of claim 1 or 2, wherein the dosage form is administered under fasted conditions.

4. The method of claim 1 or 2, wherein the dosage form is administered under fed conditions.

5. The method of any one of the preceding claims, wherein the therapeutically effective amount is equivalent to about 0.5 to about 5.0 mg of Compound 1.

6. The method of claim 5, wherein the therapeutically effective amount is in an amount equivalent to 2 mg of Compound 1.

7. The method of claim 5, wherein the therapeutically effective amount is in an amount equivalent to 3 mg of Compound 1.

8. The method of any one of the preceding claims, wherein the dosage form is administered without titration.

9. The method of claim 5, wherein the individual is administered an amount equivalent to 2 mg of Compound 1 for a first time period and subsequently an amount equivalent to 3 mg of Compound 1 for a second time period.

10. The method of any one of the preceding claims, wherein the Compound 1, or a pharmaceutically acceptable salt thereof is administered orally.

11. The method of any one of the preceding claims, wherein the Compound 1, or a pharmaceutically acceptable salt thereof, is formulated as a capsule or tablet suitable for oral administration.

12. The method of any one of the preceding claims, wherein the Compound 1, or a pharmaceutically acceptable salt thereof, is selected from:
Compound 1;
a calcium salt of Compound 1; and
an L-arginine salt of Compound 1.

13. The method of claim 12, wherein the Compound 1, or a pharmaceutically acceptable salt thereof, is an L-arginine salt of Compound 1.

14. The method of claim 13, wherein the Compound 1, or a pharmaceutically acceptable salt thereof, is an anhydrous, non-solvated crystalline form of an L-arginine salt of Compound 1.

15. The method of claim 12, wherein the Compound 1, or a pharmaceutically acceptable salt thereof, is an anhydrous, non-solvated crystalline form of Compound 1.

16. The method of any one of the preceding claims, wherein the therapeutically effective amount of Compound 1, or a pharmaceutically acceptable salt thereof, is administered once daily to the individual.

17. The method of any one of the preceding claims, wherein the method is non-gender specific.

18. The method of any one of the preceding claims, wherein the individual was previously administered at least one agent selected from: a TNFa antagonist, an integrin antagonist, and an immunosuppressive agent.

19. The method of claim 18, wherein the individual had an inadequate response with, lost response to, or was intolerant to the at least one agent.

20. The method of any one of the preceding claims, wherein treating comprises inducing and/or maintaining clinical response; improving endoscopic appearance of the mucosa; and/or inducing and/or maintaining clinical remission.

21. The method of any one of the preceding claims, wherein said administering results in no serious adverse events.

22. The method of any one of the preceding claims, wherein the Compound 1 is administered without substantially inducing an acute heart rate reduction or heart block in the individual.

23. The method of any one of the preceding claims, further comprising monitoring for adverse events during the administration of Compound 1, or a pharmaceutically acceptable salt thereof, and optionally, interrupting or terminating the administration of Compound 1, or a pharmaceutically acceptable salt thereof.

24. The method of any one of the preceding claims, wherein said individual has moderately to severely active Crohn's Disease.

25. The method of claim 24, wherein said individual has moderately active Crohn's Disease.

26. The method of claim 24, wherein said individual has severely active Crohn's Disease.

27. The method of any one of the preceding claims, wherein said individual has had inadequate response to conventional therapy.

28. The method of claim 27, wherein said conventional therapy is selected from at least one of corticosteroids, immunosuppressants, and biologics.

29. The method of claim 27, wherein said conventional therapy is selected from at least one of prednisone, budesonide, 6-mercaptopurine, azathioprine, methotrexate, infliximab, adalimumab, or certolizumab pegol.

30. The method of any one of the preceding claims, wherein said individual is not co-administered a TNFα inhibitor.

31. The method of any one of the preceding claims, wherein Compound 1 is the sole active ingredient administered to the individual for treatment of said Crohn's Disease.

32. A method of treating an individual with Crohn's Disease comprising:
administering an induction dose of Compound 1, or a pharmaceutically acceptable salt thereof, to the individual in need thereof for an induction phase, wherein the induction phase comprises at least 14 weeks; and
administering a maintenance dose of Compound 1, or a pharmaceutically acceptable salt thereof, to the individual in need thereof for a maintenance phase.

33. The method of claim 32, wherein the induction dose comprises an amount equivalent to 3 mg of Compound 1.

34. The method of any one of claim 32 or claim 33, wherein the maintenance dose comprises an amount equivalent to 2 mg of Compound 1.

35. The method of any one of claims 32-34, wherein the maintenance phase comprises at least 38 weeks.

36. The method of any one of claims 32-35, wherein the Compound 1 or a pharmaceutically acceptable salt thereof is administered at a frequency of once a day during both the induction dose and the maintenance dose.

37. The method of any one of claims 32-36, wherein said individual has moderately to severely active Crohn's disease.

38. A compound that is (R)-2-(7-(4-cyclopentyl-3-(trifluoromethyl)benzyloxy)-1,2,3,4-tetrahydrocyclopenta [b]indol-3-yl)acetic acid (Compound 1), or a pharmaceutically acceptable salt thereof, for use in a method of treatment of Crohn's Disease in an individual.

39. A compound that is (R)-2-(7-(4-cyclopentyl-3-(trifluoromethyl)benzyloxy)-1,2,3,4-tetrahydrocyclopenta [b]indol-3-yl)acetic acid (Compound 1), or a pharmaceutically acceptable salt thereof, for use in a method of treatment of Crohn's Disease in an individual, wherein the method comprises:
administering an induction dose of the compound to the individual for an induction phase, wherein the induction phase comprises at least 14 weeks; and
administering a maintenance dose of the compound to the individual for a maintenance phase.

40. The compound for use according to claim 39, wherein the induction dose comprises an amount equivalent to 3 mg of Compound 1.

41. The compound for use according to claim 39 or 40, wherein the maintenance dose comprises an amount equivalent to 2 mg of Compound 1.

42. The compound for use according to any one of claims 39-41, wherein the maintenance phase comprises at least 38 weeks.

43. The compound for use according to any one of claims 39-42, wherein the compound is administered at a frequency of once a day during both the induction dose and the maintenance dose.

44. The compound for use according to any one of claims 39-43, wherein said individual has moderately to severely active Crohn's Disease.

45. Use of a compound that is (R)-2-(7-(4-cyclopentyl-3-(trifluoromethyl)benzyloxy)-1,2,3,4-tetrahydrocyclopenta [b]indol-3-yl)acetic acid (Compound 1), or a pharmaceutically acceptable salt thereof, in the manufacture of a medicament for use in a method of treatment of moderately to severely active Crohn's Disease in an individual.

46. Use of a compound that is (R)-2-(7-(4-cyclopentyl-3-(trifluoromethyl)benzyloxy)-1,2,3,4-tetrahydrocyclopenta [b]indol-3-yl)acetic acid (Compound 1), or a pharmaceutically acceptable salt thereof, in the manufacture of a medicament for use in a method of treatment of moderately to severely active Crohn's Disease in an individual, wherein the method comprises:
administering an induction dose of the compound to the individual for an induction phase, wherein the induction phase comprises at least 14 weeks; and
administering a maintenance dose of the compound to the individual for a maintenance phase.

47. Use according to claim 46, wherein the induction dose comprises an amount equivalent to 3.0 mg of Compound 1.

48. Use according to claim 46 or 47, wherein the maintenance dose comprises an amount equivalent to 2.0 mg of Compound 1.

49. Use according to any one of claims 46-48, wherein the maintenance phase comprises at least 38 weeks.

50. Use according to any one of claims 46-49, wherein the compound is administered at a frequency of once a day during both the induction dose and the maintenance dose.

51. A method of treating an individual with Crohn's Disease comprising:
administering an induction dose of Compound 1, or a pharmaceutically acceptable salt thereof, to the individual in need thereof for an induction phase, wherein the induction phase comprises at least 14 weeks;
testing the individual for a clinical response; and
if the individual demonstrates a clinical response, administering a maintenance dose of Compound 1, or a pharmaceutically acceptable salt thereof, to the individual in need thereof for a maintenance phase.

52. The method of claim 51, wherein the induction dose comprises an amount equivalent to 3 mg of Compound 1.

53. The method of any one of claim 51 or claim 52, wherein the maintenance dose comprises an amount equivalent to 2 mg of Compound 1.

54. The method of any one of claims 51-53, wherein the maintenance phase comprises at least 38 weeks.

55. The method of any one of claims 51-54, wherein the Compound 1 or a pharmaceutically acceptable salt thereof is administered at a frequency of once a day during both the induction dose and the maintenance dose.

56. The method of any one of claims 51-55, wherein said individual has moderately to severely active Crohn's disease.

57. The method of any one of claims 51-56, wherein the clinical response is a Simple Endoscopic Score in Crohn's Disease (SES-CD) less than or equal to 4 and at least 2-point reduction from baseline with no sub-score >1.

58. The method of any one of claims 51-56, wherein the clinical response is a greater than or equal to 50% decrease from baseline in SES-CD.
